(19) United States
(12) Patent Application Publication
(10) Pub. No.: US 2005/0228465 A1
(43) Pub. Date: Oct. 13, 2005

(54) Thermal device for activatable thermochemical compositions

(76) Inventors: Christa Harris, Waukegan, IL (US); Varsha Kalyankar, Round Lake Beach, IL (US)

Correspondence Address: ALLEGIANCE CORPORATION;ATTN: Kim Luna, KB-1A, 1430 Waukegan Rd, McGaw Park, IL, 60085-6787, US

(21) Appl. No.: 10/821,338

(22) Filed: Apr. 9, 2004

(51) Int. Cl. A61F 7/00

(52) U.S. Cl. 607/114

(57) ABSTRACT

The invention described herein provides an improved thermal device having enhanced storage longevity and chemical integrity. The thermal device comprises a flexible plastic containment and activatable thermochemical liquid composition contained therein. The flexible plastic containment is constructed using a multilayer film comprising an outer polymeric barrier layer comprising an oxide coating, and an inner polymeric sealant layer. The thermal device inhibits or prevents water loss through the film, thereby enhancing the chemical integrity of the thermochemical composition within.

### FIELD OF THE INVENTION

Then invention relates to the field of medical devices. In particular, the invention pertains to an improved thermochemical pack.

### BACKGROUND OF THE INVENTION

Thermal packs, i.e., hot and cold packs, are well-known in the art. A variety of thermal pack configurations are available in a number of shapes, sizes, and with various associated structural features and attachments. One type of thermal pack are those that contain activatable thermochemical composition within a plastic film containment. Such thermal packs typically contain two or more chemical ingredients separated by a physical barrier, e.g., a rupturable plastic casing, that can be activated at time of use by combining the ingredients to produce a thermal effect. Similarly, other thermal pack structures include those which contain a thermochemical composition within a plastic film containment, together with a physical activator or “trigger” device which initiates crystallization and the resulting thermal effect.

One problem associated with prior thermal packs has been the ability to regulate or control the resulting temperature following their activation. A variety of insulation materials have been used on the exterior of thermal packs in an effort to “buffer” undesired extreme temperatures from the user's skin surface upon which the packs can be placed. Such thermal packs can contain a plastic film containment within a woven or non-woven fabric pouch. One drawback associated with the insulation layer approach is that the delivery of effective temperatures to the user can be compromised.

Another problem associated with conventional thermal packs is adverse effects of prolonged storage. Thermal packs are often stored for extended periods of time, such as in hospital supply rooms and pharmacies, prior to their activation and use. Prolonged storage longevity, however, can produce water-loss from the liquid component of the thermal pack. This water loss concentrates the remaining thermochemical ingredients, which can result in generated undesirable extreme temperatures upon their activation. This can produce discomfort and even injury to the user.

Multilayer films containing layers that inhibit or prevent water loss therethrough are known. For instance, aluminum oxide coated films and silicone coated films are known to provide barriers to prevent water loss through the film. Such films are available from Toppan G1-AU Films (Uniontown, Ohio), for example. These types of films have been used in various packaging applications, such as food packaging and storage containment.

There is a need in the field of thermal packs and therapy for a thermal pack that can be stored for prolonged periods of time yet still deliver effective temperatures to the user within a desirable temperature range. Further, there is a need for thermal packs that preserve or prolong the chemical integrity of the thermochemical composition therein.

### SUMMARY OF THE INVENTION

The invention provides an improved thermal device comprising a flexible plastic containment containing an activatable thermochemical liquid composition therein. It has been discovered that a thermal device, such as a thermal pack, can be constructed so as to afford better post-activation temperature predictability and consistency without reliance upon added insulation layers or pouches, while at the same time maintains chemical integrity of the thermochemical liquid therein over prolonged storage periods. According to the invention, the thermal device containment is constructed from a particular multilayer film that inhibits or prevents water loss therethrough. The thermal device of the invention is particularly useful in thermal packs and devices that are activated at time of use and applied onto the skin or body of the user to deliver thermal effect.

The invention provides thermal device comprising a flexible plastic containment and activatable thermochemical liquid composition therein, said flexible plastic containment comprising a multilayer film comprising: an outer polymeric barrier layer comprising an oxide coating; and an inner polymeric sealant layer. In a preferred embodiment, the two film layers are laminated together by an adhesive layer between.

### BRIEF DESCRIPTION OF THE DRAWINGS

FIG. 1 is an angled side view of a thermal device constructed using a multiplayer film according to one embodiment of the invention.

FIG. 2 is a cross-sectional view of a thermal device showing the film construction and contents of the device according to one embodiment of the invention.

FIG. 3 is a cross-sectional view of the multilayer film used in the device according to one embodiment of the invention.

### DETAILED DESCRIPTION OF THE INVENTION

As used herein, the phrase “topically-applied” within the context of the thermal device of the invention is meant to refer to the localized delivery of thermal treatment to the user's body. For example, the phrase can refer to thermal delivery to an area of the body, e.g., heel, knee, and the like, by application of the device adjacent to the skin surface.

The thermal device according to the invention can be used to deliver hot or cold therapies to the user's skin. For illustrative purposes, the thermal device of the invention is described in terms of delivering warming therapy through the use of activatable exothermic thermochemical formulations.

The thermal device of the invention can be constructed using a variety of shapes, sizes and configurations, depending upon their contemplated or intended application. The overall configuration of the device can be selected to conform with the anatomical geometry of a particular site on the body. The thermal device can be configured, for example, for application to, or attachment onto, the user's knee, elbow, heel, ankle, neck, shoulder, chest, stomach, back, hip, and the like.

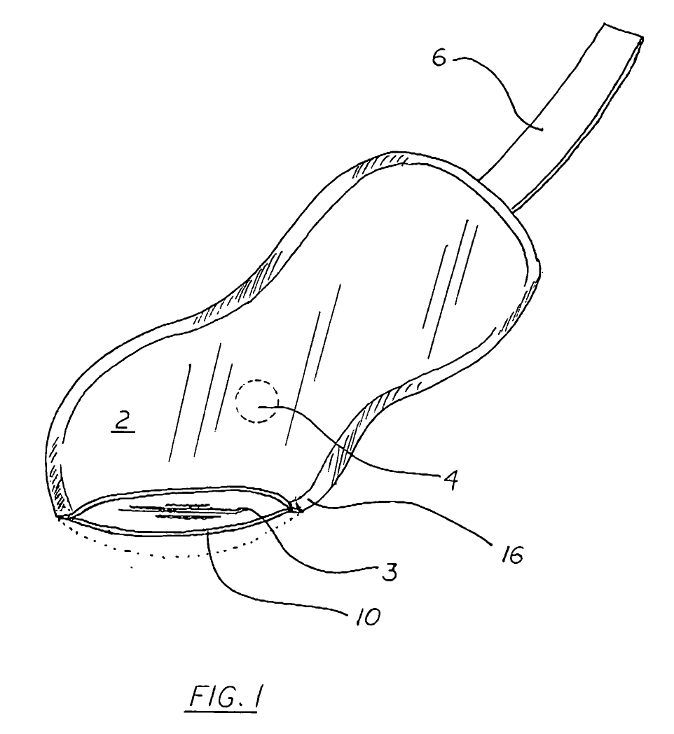

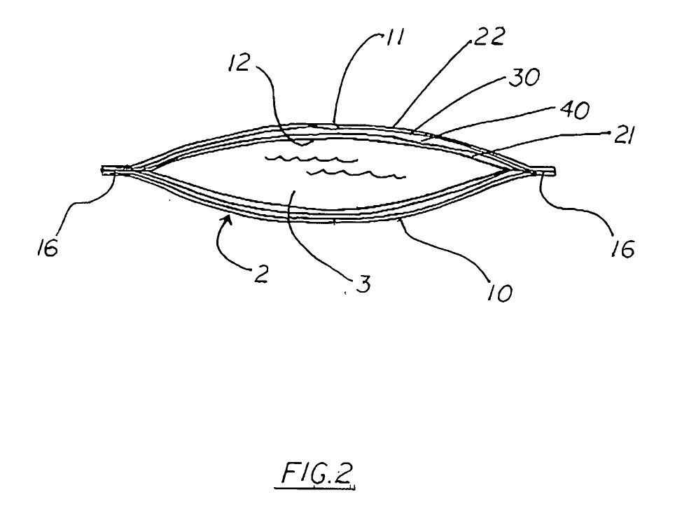

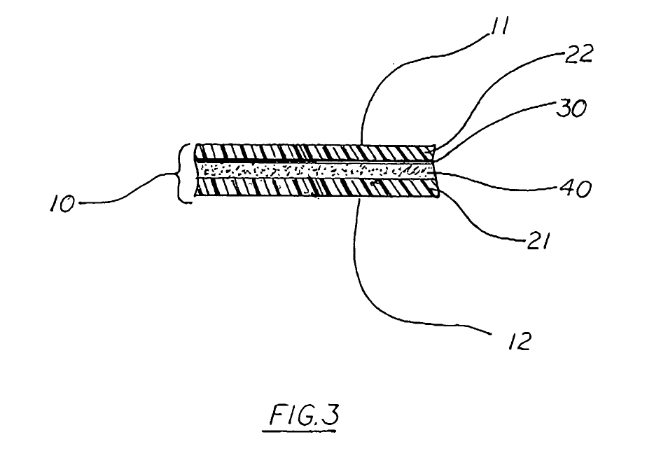

FIG. 1 illustrates one embodiment of the invention in the form of an infant heel warming device, including a strap 6 to secure the device in place during thermal delivery to the user. In general, the thermal device of the invention comprises a flexible plastic containment 2 containing an activatable thermochemical composition 3 therein and physical activator element 4. The flexible plastic containment 2 comprises a multilayer film 10 which separates and forms a barrier between the interior liquid/chemical environment of the device and the exterior environment. The multilayer film, therefore, has an outer surface 11 in contact with the external environment, and an inner surface 12 in contact with the liquid internal contents. (See FIGS. 2 and 3, for example).

The improved effectiveness of this barrier formed by the multilayer film is an important aspect of the invention. The multilayer film of the containment of the device functions more than a mere liquid-proof containment for a liquid composition encased therein. As topically-applied thermal devices are typically stored for prolonged periods of time after which they are activated and applied to the user's skin to effect thermal therapy, the preservation of the thermochemical chemical composition is critical to the temperature delivered upon its activation. Gradual evaporative water loss from the interior environment and thermochemical composition through the containment material can adversely affect the activation temperature when the device is activated at a later date. For example, undesirable exothermic temperature extremes can be produced by reduced concentrations of water in certain thermochemical compositions, which can in turn cause discomfort or injury to the user.

Thermochemical compositions susceptible to ingredient loss during storage include, for example, aqueous sodium acetate formulations, aqueous sodium thiosulfate formulations, and aqueous magnesium sulfate formulations. Any activatable thermochemical formulation comprising a supersaturated aqueous mixture containing a chemical salt can benefit from advantages afforded by the thermal device of the invention. Sodium acetate formulations have, however, been found to be particularly susceptible to changes in concentration due to water loss, i.e., the slope of temperature versus concentration curve is relatively steep. Thermochemical compositions such as aqueous sodium acetate can be activated at time of use by physical activator elements, also referred to as “triggers” or “clickers” in the art. Such activator elements can be in the form of flexible perforated metallic discs or crystal-coated boards or papers. Such thermochemical activator compositions and physical activators are described in Applicant's own U.S. patent application Ser. No. ______ (not yet assigned), filed concurrently herewith, the full text of which is incorporated herein by reference.

Referring now to FIGS. 2 and 3, the multilayer film 10 used in accordance with the invention generally comprises an inner polymeric sealant layer 21 and outer polymeric barrier layer 22, wherein the outer polymeric barrier layer comprises an oxide coating 30 on at least one side. The inner polymeric sealant layer 21 can be composed of one or more materials provided that the desired seal strength and integrity is afforded to the layer to contain a liquid. Suitable polymeric materials that can be used for the inner sealant layer 21 include polyolefins, such as ethylvinyl acetate, polyethylene, polypropylene, and the like. In one embodiment, the inner polymeric sealant layer is composed of an ethylvinyl acetate (EVA)/polyethylene blend. Preferably, the polyethylene used in the blend is low density polyethylene (LDPE).

The outer polymeric barrier layer 22 further comprises a high barrier coating 30 on at least one side, preferably the interior-oriented surface the barrier layer. The coated outer polymeric barrier layer 22 is positioned as the outward-oriented side of the device containment 2, e.g., the patient contacting side. The outer polymeric barrier layer 22 can be composed of a polymeric material that can be coated with an oxide coating, e.g., aluminum oxide or silicone oxide, to form a vapor barrier in accordance with the invention. Suitable outer polymeric barrier layer materials include, but are not limited to, polyester. A preferred polymeric material for the outer barrier layer is polyester because of its receptivity to aluminum oxide surface coating techniques. The high barrier coating 30 thereon can be composed of an oxide compound. Preferred oxide coatings that can be used include aluminum oxide and silicone oxide. Most preferred is an aluminum oxide coating. In a preferred embodiment, the outer polymeric barrier layer can be composed of aluminum oxide-coated polyester film having a water/vapor transmission date (WVTR) of about 0.12 g/m²/day (at 100 F with 90% relative humidity) or less.

The outer barrier layer 22 and inner sealant layer 21 are preferably laminated together by an adhesive layer 40 positioned between as shown in FIG. 3. A variety of adhesive compositions can be used to accomplish the lamination of the outer and inner film layers. In one embodiment, the adhesive layer comprises ethyl acetate/aromatic polyisocyanate/methyl acetate blend. Collectively, the coated outer barrier layer 22 (with 30) and inner sealant layer 21 adhered together form the multilayer film 10 of the containment 2.

When selecting materials for the outer barrier layer and inner sealant layer, the perimeter sealing technique should be considered. For example, when heat sealing is used to seal the containment, the material used for the inner sealant layer should have a lower melting point range than the materials used for the outer barrier layer so as to facilitate the formation of the sea as part of the manufacturing process.

The thermal device according to the invention can be made using conventional equipment and techniques readily available to those skilled in the thermal pack field. In general, the containment itself can be constructed as a thermal sealed bag, or two aligned sheets that are sealed about their perimeter 16 (as shown in FIGS. 1 and 2). The thermal device of the invention can be assembled by heat sealing layers of flexible polymeric films around a predetermined liquid fill volume of the thermochemical composition with an activator element deposited therein. Once the containment has been made, securing means such as straps (illustrated as numerical reference 6 in FIG. 1) can be affixed to an exterior surface location of the device in accordance with the anticipated use of the device. In a further embodiment (not shown), a layer of non-woven material can be laminated onto the outer surface of the containment on one or both sides of the device, such as described in Applicant's own U.S. patent application Ser. No. ______, filed concurrently herewith and co-pending, the text of which is incorporated herein by reference.

In use, the thermal device of the invention is activated by initiating the thermochemical reaction of the encased composition. This can be accomplished by combining two or more ingredients physically separated from one another until time of use, or by initiating a chemical reaction by a physical activator element (illustrated as numerical reference 4 in FIG. 1), such as a trigger or crystallization initiator. Once commencement of the thermal effect is confirmed, the device can be placed against the portion of the user's body or skin to which delivery of the thermal effect is desired.

### INDUSTRIAL APPLICABILITY

The invention is useful in the medical field for delivery of thermal therapy to a patient's skin or portion of the user's body. Such thermal therapy can be in the form of either warming or cooling temperatures associated with treating or comforting traumatized tissues.

The invention has been described herein above with reference to various and specific embodiments and techniques. It will be understood by one of ordinary skill, however, that reasonable modifications and variations can be made from such embodiments and techniques while remaining within the spirit and scope o the invention as defined by the claims set forth below.

1. A topically-applied thermal device comprising a flexible plastic containment and activatable thermochemical liquid composition encased therein, said flexible plastic containment comprising a multilayer film comprising:
an outer polymeric barrier layer comprising an oxide coating; and
an inner polymeric sealant layer.

2. The thermal device according to claim 1, further comprising an adhesive layer laminating together said outer polymeric barrier layer and said inner polymeric sealant layer.

3. The thermal device according to claim 1, wherein said oxide coating is selected from the group consisting of aluminum oxide and silicone oxide.

4. The thermal device according to claim 1, wherein said outer polymeric barrier layer comprises polyester.

5. The thermal device according to claim 1, wherein said inner polymeric sealant layer comprises a blend of low density polyethylene (LDPE) and ethylvinyl acetate (EVA).

6. A topically-applied thermal device comprising a flexible plastic containment and activatable thermochemical liquid composition encased therein, said flexible plastic containment comprising a multilayer film comprising:
an outer polymeric barrier layer comprising polyester, said outer layer further comprising an oxide coating on the interior surface of said polyester layer;
an inner polymeric sealant layer comprising a blend of low density polyethylene (LDPE) and ethylvinyl acetate (EVA); and
an adhesive layer between said outer layer and said inner layer.

7. The thermal device according to claim 6, wherein said oxide coating comprises aluminum oxide.

8. The thermal device according to claim 6, wherein said thermochemical liquid composition comprises a supersaturated aqueous mixture comprising a chemical salt.

9. The thermal device according to claim 8, wherein said thermochemical liquid composition comprises aqueous sodium acetate; aqueous sodium thiosulfate; or aqueous magnesium sulfate.

10. The thermal device according to claim 6, wherein said adhesive layer comprises an ethyl acetate/aromatic polyisocyanate/methyl acetate blend.
